(19) United States
(12) Patent Application Publication
(10) Pub. No.: US 2010/0166895 A1
(43) Pub. Date: Jul. 1, 2010

(54) Capsicum extract for treatment of skin cancer

(76) Inventor: Francisco Silviera Louro, Weston, FL (US)

Correspondence Address: Rogers IP Group, Church Street Station, P.O. Box 3115, New York, NY, 10008-3115, US

(21) Appl. No.: 12/721,507

(22) Filed: Mar. 10, 2010

(30) Foreign Application Priority Data
Sep. 27, 2007 (US) PCT/US2007/020775

(51) Int. Cl. A61K 36/81; A61P 35/00

(52) U.S. Cl. 424/760

(57) ABSTRACT

A process for the treatment of skin cancer which includes topically applying to an affected area of a human patient in need of such treatment a therapeutically effective amount of a capsicum extract. The capsicum extract comprises a fresh raw Capsicum fruit and a carrier. In one preferred embodiment, the Capsicum fruit is a pepper such as a habanero chili pepper and the carrier is alcohol, corn oil, lanolin or vegetable oil.

### CROSS-REFERENCE TO RELATED APPLICATIONS

This application claims priority to International Application PCT/US2007/020775, with an international filing date of Sep. 27, 2007 published in English under PCT Article 21(2).

### BACKGROUND OF THE INVENTION

1. Technical Field

The present invention relates to a method of treating skin cancer, and more particularly to a method of using a capsicum extract to treat basal and squamous cell carcinoma.

2. Background

Skin cancer is the most common form of cancer in the United States. Skin cancers are classified by the types of epidermal cells involved. Basal cell carcinoma (BCC) develops from abnormal growth of the cells in the lowest layer of the epidermis. BCC is the most common form of skin cancer, affecting about one million Americans each year. Squamous cell carcinoma (SCC) involves changes in the squamous cells, found mainly in the middle layer of the epidermis. Melanoma occurs in the melanocytes (cells that produce pigment) and is less common than squamous or basal cell carcinoma, but more dangerous.

The diagnosis of skin cancers such as BCC is confirmed with a biopsy. Choice of treatment is based on the type, size, location, and depth of penetration of the tumor, the patient's age and general health. Treatment includes surgery to remove the tumor, radiation, cryosurgery as well as topical medications such as imiquimod and 5-fluorouracil (5-FU). Although many of these treatments can be successful, there is a continued need for other treatment options, particularly those with few side effects.

The Genus Capsicum is the name of a group of annual plants in the nightshade (Solanaceae) family. They are native to Mexico and Central America but are cultivated for food in many warmer regions of the world. Capsicum varieties include the cayenne pepper and other hot peppers. C. annum forms constitute the major commercial peppers in the United States. Historically, the cultivation of capsicum, and subsequent manufacture of capsicum products, for commercial use in culinary, medicinal, and repellent formulations has been primarily based on capsaicin content. Thus either for historical reasons or based on a belief that capsaicin is the major active ingredient in the Capsicum fruit, comparatively little research has been reported using the Capsicum fruit as an anti-cancer agent (American Cancer Society Complete Guide to Complementary & Alternative Cancer Therapies, Second Edition, 2009).

U.S. Pat. No. 6,235,788 to Terry discloses treating pre-malignant basal and squamous cell lesions of the epithelium by applying a certain concentration of capsaicin in a base of Aloe vera to an area of the lesion. To the knowledge of the inventors, however, no prior art exists which discloses the use of capsaicin for treating malignant rather than pre-malignant basal and squamous cell lesions of the epithelium. As with most research relating to Capsicum, Terry solely discloses the use of capsaicin in a method of treating pre-malignant basal and squamous cell lesions. In contrast, the inventor of the current invention has discovered that an extract using fresh raw Capsicum fruit along with a carrier is a surprisingly effective method of healing forms of skin cancer such as basal cell lesions.

### SUMMARY OF INVENTION

A novel process for the treatment of skin cancer which includes topically applying to an affected area of a human patient in need of such cancer treatment a therapeutic amount of a capsicum extract. The capsicum extract comprises a mixture of fresh raw Capsicum fruit and a carrier. In one preferred embodiment of the invention, the Capsicum fruit is a pepper such as a habanero chili pepper. Examples of carriers according to the present invention include alcohol, lanolin and vegetable oils. In one aspect of the invention, the capsicum extract will have a therapeutically effective amount of capsaicin which is obtained by increasing or decreasing the amount of Capsicum fruit in the extract.

### BRIEF DESCRIPTION OF THE DRAWINGS

There are no drawings in this application.

### DETAILED DESCRIPTION OF THE INVENTION

### 1. Definitions

The term “Capsicum” is the name of a group of annual plants in the nightshade (Solanaceae) family. They are native to Mexico and Central American but are cultivated for food in many warmer regions of the world. Capsicum varieties include the cayenne pepper; jalapeño pepper, other hot peppers, and paprika (American Cancer Society Complete Guide to Complementary & Alternative Cancer Therapies, Second Edition, 2009). The name Capsicum comes from the Greek kept, which means “to bite”.

The term “capsaicin” is the most-studied active ingredient in the Capsicum plant (American Cancer Society Complete Guide to Complementary & Alternative Cancer Therapies, Second Edition, 2009). After early controversy, capsaicin was found to be a mixture of similar compounds. Capsaicin is available commercially from a variety of suppliers such as the Sigma Chemical Company.

The term “capsaicinoids” is used to indicate capsaicin and its analogs. Capsaicinoids include the following chemical structures, (1) Capsaicin (60 to 65% by weight), (2) Nordihydrocapsaicin, (3) Nonylvanillylamide, (4) Dihydrocapsaicin, (5) Decanylvanillylamide and (6) Homocapsaicin. A total amount of capsinoids in the commercial capsaicin is more than 95% by weight and a main component of the commercial capsaicin, Capsaicin, is about 60 to about 65% by weight (See for example, U.S. Pat. No. 5,955,631, which is incorporated by reference in its entirety herein).

The term Capsicum fruit of fruit of the Capsicum plant is referred to as ovary which is swelled after the floral organ is pollinated and contains the next-generation seed. The cavity accounts for most inside of the fruit, except for the seed, the placenta, and the dissepiments, and therefore, fleshy fruit is eaten.

Peppers are members of the genus Capsicum and family Solanaceas, which includes the species Capsicum annuum and Capsicum frutescens. Many varieties of the Capsicum species are not hot or pungent. Pungency depends on the content of capsaicin or its related substances, capsaicinoids. Commercial peppers are diploids with n=12 chromosomes. Peppers are cultivated and used around the world as sweet peppers such as the bell pepper; or as pungent chili peppers, jalapeno peppers, and TABASCO peppers; or as a source of dried powders of various colors such as paprika. The types of cultivated peppers can be differentiated by pungency, fruit shape, color and size (See for example U.S. Pat. No. 6,498,287, which is incorporated herein by reference).

### 2. Preparation and Use of Capsicum Extract

The capsicum extract of the present invention is prepared from a fresh raw fruit from the Capsicum plant in combination with a carrier. In a preferred embodiment of the present invention, the Capsicum plant comes from the species Capsicum annuum or Capsicum frutescens. In one particularly preferred embodiment, the Capsicum fruit is a pepper, such as a habanero chili pepper.

Various carriers can be used in conjunction with the present invention. In one preferred embodiment, the carrier is alcohol. In another preferred embodiment, the carrier is corn oil. In yet another preferred embodiment, the carrier is lanolin.

The individual components of the composition may be mixed using any method known in the art to form the composition. In one preferred embodiment of the invention, the components are mixed with a blender. After the composition is thoroughly mixed, the composition is next left to sit, preferably in a tight container. In one preferred embodiment, the composition is left to sit for at least three days prior to use of the now prepared capsicum extract.

A fundamental aspect of the inventor's invention which distinguishes it from other attempts to treat cancer is the use of a fresh raw Capsicum fruit in the extract. Other research using Capsicum for cancer treatment typically, for historical or other reasons, focus solely upon the use of capsaicin, which is believed to be the active ingredient in Capsicum.For example, U.S. Pat. No. 6,235,788 to Terry discloses treating pre-malignant basal and squamous cell lesions of the epithelium by applying a certain concentration of capsaicin in a base of Aloe vera to an area of the lesion. It does not disclose the use of a fresh raw Capsicum fruit in a carrier as does the current inventor. Terry also discloses treating pre-malignant lesions whereas the extract of the current invention can be used for treating cancerous lesions. The inventor has performs experiments on human subjects which proves that the extract of the current invention cures basal and cells carcinoma lesions (see Example section below). It is believed by the current inventor that the success obtained using the capsicum extract of the current invention for the treatment of skin cancer is due to a blend of constituents contained within a fresh raw Capsicum fruit. Without being bound by any particular theory, it appears that other constituents contained within the capsicum extract besides or in addition to capsaicin may be partially or wholly responsible for the observed therapeutic effects of the capsicum extract of the present invention. It is also essential that the Cap sum fruit used in preparation of the fruit is a raw fresh fruit.

In addition to the inventor's own success in treating cancerous cell lesions of the epithelium with the extract of the present invention, another advantage of using the entire Capsicum fruit according to the present invention is that it avoids the pungency effects of using capsaicin directly. Capsaicin is well known to cause burning when it applied directly to mucous membranes. In high concentrations, it will also cause a burning effect to other sensitive areas of the skin. As a result of its pungency, capsaicin is an active ingredient in pepper sprays used for personal defense as well as to deter rodents. Although not to be bound by any theory, the inventor believes that other ingredients in the Capsicum plant counteract the pungent effects of using capsaicin alone. By using the Capsicum extract of the current invention, one is thus able to avoid the potentially toxic effects of capsaicin. There is also no need to include other constituents such as a local pain killer to offset the irritating effects of capsaicin which is a strategy used by other inventors in therapeutic compositions containing capsaicin (see for example, U.S. Pat. No. 6,284,797).

Capsicum fruit will naturally contains a percentage of capsaicin and in fact capsaicin may be an important constituent in the Capsicum extract of the current invention. There is considerable research which can substantiate anti-cancer properties of capsaicin, and the inventor is not discounting the possibility that capsaicin plays a role in the observed effects of the capsicum extract of the present invention. Some proponents claim that capsaicin has antioxidant properties that help to fight nitrosamine, a cancer-causing agent. Still others claim that capsaicin may prevent DNA damage and lung cancer due to cigarette smoke (American Cancer Society Complete Guide to Complementary & Alternative Cancer Therapies, Second Edition, 2009). Several studies have also shown that capsaicin may be useful for managing side effects of conventional cancer therapy such as pain related to surgery and mouth sores due to chemotherapy and radiation therapy.

As a result of the probably beneficial properties of capsaicin, in one preferred embodiment, capsaicin content in the capsicum extract is adjusted to a level so as to provide the extract with its most therapeutic effect. Because Capsicum fruit naturally contains capsaicin, the level of capsaicin can be corresponding increased or decreased according to the amount of Capsicum fruit which is ground up to make the capsicum extract. The percentage of capsaicin content of peppers may be measured using any method known in the art. The Scoville rating, for example, is one method to measure the capsaicin content of peppers. Dividing the Scoville rating of a pepper by fifteen (15) gives the parts per million (ppm) capsaicin content of that specific pepper.

The capsicum extract of the present invention is topically applied to affected areas of a patient.

### Example 1

### Preparation of Capsicum Extract

Fresh raw Habanero chili peppers were purchased locally from Publix supermarket and sliced. The sliced peppers were next added to a jar in the amount of two times the weight of alcohol. The contents of the jar were next mixed and blended using a commercial blender. The jar was sealed, and the mixture was then left for 3 weeks whereupon the peppers would lose their color and sink to the bottom of the jar. Next, the contents of the jar was filtered into a second jar, now containing the Capsicum extract.

### Example 2

### Case History 1

A patient was diagnosed by a physician (dermatologist) as having basal and squamous cell lesions of the epithelium. A treatment course was commenced consisting of spraying the cancerous lesions which had appeared on the face and scalp of the patient with liquid nitrogen. Five cancerous lesions were also surgically removed from the patient's neck and face. Despite the conventional treatment, cancerous lesions continued to reappear on the patient. An extract of Capsicum as prepared in Example 1 was thereafter topically applied to two cancerous moles which reappeared on each of the patient's breasts, close to the nipples. Several minutes after application, the cancerous moles shrunk. The following day, the lesion had developed into a scab. The third day, the scab peeled off, and the skin had regenerated to its original form. The same extract was applied to other lesions which appeared on the patient with the same result. For 2.5 years, the same patient has been using the same extract on other lesions with the same result. In every single case, the capsicum extract has healed the lesions and the patient has not needed to undergo any conventional skin cancer treatment during these 2.5 years.

### Example 3

### Case History 2

A patient was diagnosed with basal and squamous cell lesions of the epithelium by a physician. The patient had two lesions on his left arm which persistently reappeared despite conventional liquid nitrogen treatment. An extract of Capsicum as prepared in Example 1 was thereafter applied to the lesions. The lesions disappeared in a week.

The principles, preferred embodiments and modes of operation of the present invention have been made apparent in the foregoing description. The invention should not be construed as limited to the described embodiments. Instead, the embodiments described herein should be regarded as illustrative and not restrictive in any way. Variations and changes may be made by others without departing from the intent of the present invention as defined by the following claims:

What is claimed is:

1. A process for treating skin cancer, comprising: topically applying to an affected area of a human patient in need of such treatment a therapeutically effective amount of a capsicum extract, said extract comprising a fresh raw Capsicum fruit and a carrier.

2. The process of claim 1, wherein said skin cancer is selected from a group consisting of basal cell carcinoma and squamous cell carcinoma.

3. The process of claim 2, wherein said Capsicum fruit comprises a pepper and said carrier comprises alcohol.

4. The process of claim 3, wherein said pepper comprises a habanero chili pepper.

5. The process of claim 4, wherein said capsicum extract further comprises capsaicin.

6. The process of claim 2, wherein said Capsicum fruit comprises a pepper and said carrier comprises corn oil.

7. The process of claim 6, wherein said capsicum extract further comprises a therapeutically effective amount of capsaicin.

8. The process of claim 1, wherein said Capsicum fruit is present in a 2 to 1 weight ratio to said carrier.

9. A process for treating skin cancer, comprising: topically applying to an affected area of a human patient in need of such treatment a therapeutically effective amount of a capsicum extract, said extract consisting essentially of a Capsicum fruit and a carrier.

10. The process of claim 9, wherein said skin cancer is selected from a group consisting of basal cell carcinoma and squamous cell carcinoma.

11. The process of claim 10, wherein said Capsicum fruit comprises a pepper and said carrier comprises alcohol.

12. The process of claim 11, wherein said pepper comprises a habanero chili pepper.

13. The process of claim 12, wherein said capsicum extract further comprises a therapeutically effective amount of capsaicin.

14. The process of claim 10, wherein said Capsicum fruit comprises a pepper and said carrier comprises corn oil.

15. The process of claim 14, wherein said capsicum extract further comprises a therapeutically effective amount of capsaicin.

16. The process of claim 9, wherein said Capsicum fruit is present in a 2 to 1 weight ratio to said carrier.

17. A process for treating skin cancer, comprising: topically applying to an affected area of a human patient in need of such treatment a therapeutically effective amount of a capsicum extract, said extract comprising a fresh raw habanero chili pepper and a carrier, said habanero chili pepper being present in excess by weight to said carrier.

18. The process of claim 17, wherein said capsicum extract further comprises capsaicin.

19. The process of claim 18, wherein said carrier is selected from the group consisting of alcohol, corn oil, lanolin and vegetable oil.
